(19) United States
(12) Patent Application Publication
(10) Pub. No.: US 2022/0114916 A1
(43) Pub. Date: Apr. 14, 2022

(54) HEART SIMULATOR

(71) Applicant: ASAHI INTECC CO., LTD., Seto-shi (JP)

(72) Inventors: Satoshi NAMIMA, Seto-shi (JP); Masakazu NAKADA, Seto-shi (JP)

(73) Assignee: ASAHI INTECC CO., LTD., Seto-shi (JP)

(21) Appl. No.: 17/558,623

(22) Filed: Dec. 22, 2021

Related U.S. Application Data
(63) Continuation of application No. PCT/JP2020/022494, filed on Jun. 8, 2020.

(30) Foreign Application Priority Data
Jul. 5, 2019 (JP) 2019-125905

(51) Int. Cl. G09B 23/28; A61B 90/00

(52) U.S. Cl. CPC G09B 23/286; A61B 90/00

(57) ABSTRACT

A heart simulator includes: a heart model simulating the heart and having a cardiac apex portion and a cardiac base portion; a cardiovascular model arranged outside the heart model; and a pericardium member covering the heart model and the cardiovascular model. The pericardium member has a plurality of through-holes that penetrate the inside and the outside of the pericardium member.

### CROSS REFERENCE TO RELATED APPLICATIONS

The present application claims priority to international application PCT/JP2020/022494, filed Jun. 8, 2020, which claims the priority of Japanese Patent Application No. 2019-125905 filed on Jul. 5, 2019, the entire disclosure of both of which are incorporated herein by reference.

### TECHNICAL FIELD

The present disclosure relates to a heart simulator.

### BACKGROUND ART

Medical devices such as catheters are used for low invasive treatments or examinations of living-body inner cavities such as the circulatory system and the digestive system For example, patent literatures 1 to 5 disclose simulators (a simulated human body or a simulated blood vessel) with which an operator such as a medical practitioner can simulate procedures using these medical devices.

### CITATION LIST

### Patent Literature

[Patent Literature 1] Japanese Patent Application Laid-Open No. 2012-68505

[Patent Literature 2] Japanese Patent Application Laid-Open No. 2012-203016

[Patent Literature 3] Japanese Patent Application Laid-Open No. 2014-228803

[Patent Literature 4] Japanese Patent Application Laid-open (Translation of PCT Application) No. 2004-508589

[Patent Literature 5] Japanese Patent Application Laid-Open No. 2017-40812

When treatment or examination is performed using a catheter, angiography may be used in order to determine hemodynamics such as blood flow velocity and blood viscosity or an occluded state of a blood vessel. In angiography, a contrast agent with low radiolucency is injected through a catheter inserted into a blood vessel to perform radiography. An operator can determine hemodynamics and vascular conditions by investigating changed contrast in the resulting X-ray images (still pictures or video) to elucidate how the contrast agent flows.

For this reason, the flow of a contrast agent in a simulator (a simulated human body or a simulated blood vessel) needs to be as similar as possible to the actual flow in the living body. To this end, in a simulated human body described in Patent Literatures 1 and 2 where a simulated left coronary artery and a simulated right coronary artery are connected to a reservoir space inside a heart model, a contrast agent is diluted in the reservoir space. However, the technology described in Patent Literatures 1 and 2 has a problem in that dilution of a highly concentrated contrast agent is time-consuming. Meanwhile, in a simulator described in Patent Literature 3, a contrast agent is directed to a flow path formed to have a shape which simulates a vein. However, the technology described in Patent Literature 3 has a problem in that a contrast agent flows into a flow path without being diluted, resulting in images which do not reflect actual conditions depending on observation angles. Meanwhile, Patent Literatures 4 and 5 do not even consider use of a contrast agent in the blood simulate.

### SUMMARY

Embodiments are directed to solving, at least partially, at least one of the above and other problems, and can be implemented according to the following aspects.

(1) According to one aspect of the present invention, provided is a heart simulator. The heart simulator includes: a heart model simulating the heart and having a cardiac apex portion and a cardiac base portion; a cardiovascular model arranged outside the heart model; and a pericardium member covering the heart model and the cardiovascular model, wherein the pericardium member has a plurality of through-holes that are formed to penetrate the inside and the outside of the pericardium member.

According to this configuration, the heart simulator includes a pericardium member that covers the heart model and the cardiovascular model and has a plurality of through-holes that are formed to penetrate the inside and the outside. Therefore, a contrast agent discharged from the cardiovascular model is gently diluted in a ripple pattern in the internal space of the pericardium member (the space inside the pericardium member and the space outside the heart model and the cardiovascular model), and then spreads and is discharged from the internal space of the pericardium member to the outside of the pericardium member through a plurality of through-holes. As a result, the heart simulator of this configuration enables the flow of a contrast agent (X-ray images) during use of the contrast agent to simulate the actual living body where a contrast agent spreads along arterioles on the surface of the heart, and then diffuses over arterioles to disappear.

(2) In the pericardium member of the heart simulator according to the above aspect, the opening area of each of the through-holes may gradually increase from the position where the pericardium member covers the cardiac apex portion of the heart model toward the cardiac base portion.

In the actual human body, arterioles on the surface of the heart, venules, and capillary vessels gradually thicken from the cardiac apex portion to the cardiac base portion, so that a relatively large amount of contrast agent spreads and disappears on the side of the cardiac base portion. According to this configuration, the opening area of each through hole of the pericardium member gradually increases from the position where the pericardium member covers the cardiac apex portion of the heart model toward the cardiac base portion. Therefore, the amount of the contrast agent that spreads and is then discharged from the pericardium member to the outside can be gradually increased from the cardiac apex portion to the cardiac base portion, as in the actual human body.

(3) In the pericardium member of the heart simulator according to any one of the above aspects, the plurality of through-holes are arranged on a concentric circle centered at the position where the pericardium member covers the cardiac apex portion of the heart model, wherein the number of the plurality of through-holes arranged concentrically may gradually increase from the position where the pericardium member covers the cardiac apex portion of the heart model toward the cardiac base portion.

In the actual human body, arterioles on the surface of the heart, venules, and capillary vessels are laid out in a mesh pattern over the surface of the heart. According to this configuration, the plurality of through-holes of the pericardium member are arranged on a concentric circle centered at the position where the pericardium member covers the cardiac apex portion of the heart model, enabling the flow of the contrast agent to simulate the actual living body where a contrast agent spreads and is then discharged from the pericardium member to the outside. Further, the number of the plurality of through-holes arranged concentrically gradually increases from the position where the pericardium member covers the cardiac apex portion of the heart model toward the cardiac base portion. Therefore, the amount of the contrast agent that spreads and is then discharged from the pericardium member to the outside can be gradually increased from the cardiac apex portion to the cardiac base portion, as in the actual human body.

(4) In the heart simulator according to of any one of the above aspects, the pericardium member has a plurality of regions having different densities of the plurality of through-holes, and, specifically, the pericardium member may be provided with regions where the opening area of the plurality of through-holes is smaller than that of the plurality of through-holes provided at the cardiac base portion and the densities of the through-holes are relatively high, at the position on the cardiac apex portion side of the heart model.

In the actual human body, arterioles on the surface of the heart, venules, and capillary vessels, specifically, each distal end of the arterioles and venules (ends on the cardiac apex portion side) is connected by capillary vessels on the cardiac apex portion side. According to this configuration, the pericardium member is provided with regions where the opening area of the plurality of through-holes is smaller than that of the plurality of through-holes provided at the cardiac base portion, and the densities of the through holes are relatively high, at the position on the cardiac apex portion side of the heart model. Therefore, the capillary vessels on the surface of the heart can be simulated by the regions, enabling the flow of the contrast agent during use of the contrast agent to further simulate the actual living body.

(5) In the heart simulator according to any one of the above aspects, the pericardium member may be formed of a thin film having elasticity lower than that of the heart model.

According to this configuration, since the pericardium member is formed with a thin film having elasticity lower than that of the heart model, a plurality of through-holes can be easily formed in/on the pericardium member.

(6) In the heart simulator according to any one of the above aspects, the pericardium member is formed of a porous body, and the plurality of through-holes may be pores of the porous body.

According to this configuration, since the pericardium member is formed of a porous body, the pores of the porous body can be used as a plurality of through-holes. Therefore, the pericardium member can be easily formed.

(7) In the heart simulator according to any one of the above aspects, the blood simulate discharged from the cardiovascular model may be discharged to the outside through the plurality of through-holes.

According to this configuration, the blood simulate discharged from the cardiovascular model is discharged to the outside through a plurality of through-holes, enabling the flow of the contrast agent during use of the contrast agent to simulate the actual living body where a contrast agent spreads along the arterioles on the surface of the heart, and then diffuses over the venules to disappear.

It is noted that the present invention can be implemented according to various aspects. For example, the present invention can be implemented according to the following aspects: a pericardium member to be used for a heart simulator; a heart simulator including a heart model, a cardiovascular model, and a pericardium member; a human body simulating apparatus including at least a part of these; and a method of controlling the human body simulating apparatus.

### BRIEF DESCRIPTION OF DRAWINGS

FIG. 1 shows a schematic configuration of a human body simulating apparatus.

FIG. 2 shows a schematic configuration of the human body simulating apparatus.

FIG. 3 shows a schematic configuration of an aortic model.

FIG. 4 shows a schematic configuration of a model.

FIG. 5 shows a schematic configuration of a model.

FIG. 6 shows a schematic configuration of a heart simulator.

FIG. 7 shows a schematic configuration of the heart simulator.

FIG. 8 illustrates a configuration of a pericardium member.

FIG. 9 illustrates a configuration of a pericardium member according to a second embodiment.

FIG. 10 illustrates a configuration of a pericardium member according to a third embodiment.

FIG. 11 illustrates a configuration of a pericardium member according to a fourth embodiment.

FIG. 12 illustrates a configuration of a pericardium member according to a fifth embodiment.

FIG. 13 illustrates a configuration of a pericardium member according to a sixth embodiment.

FIG. 14 shows a schematic configuration of a heart simulator according to a seventh embodiment.

FIG. 15 shows a schematic configuration of a heart simulator according to an eighth embodiment.

### DETAILED DESCRIPTION

Embodiments are directed to providing a heart simulator enabling the flow of a contrast agent during use of the contrast agent to simulate the actual living body.

### First Embodiment

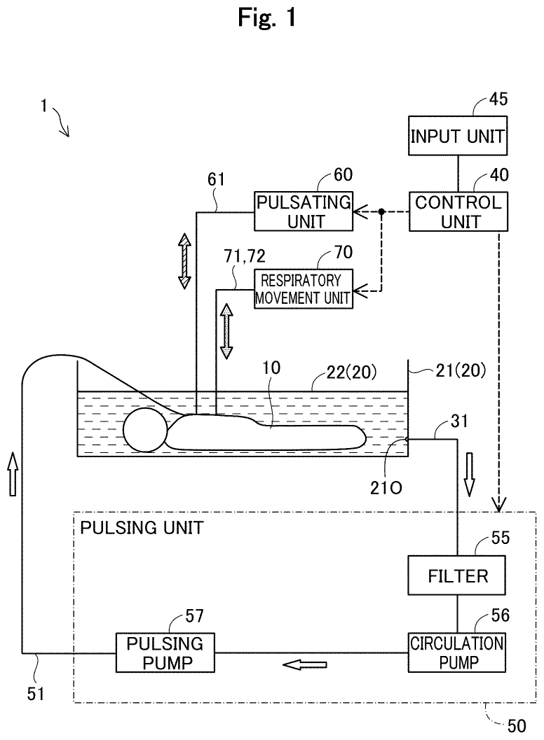

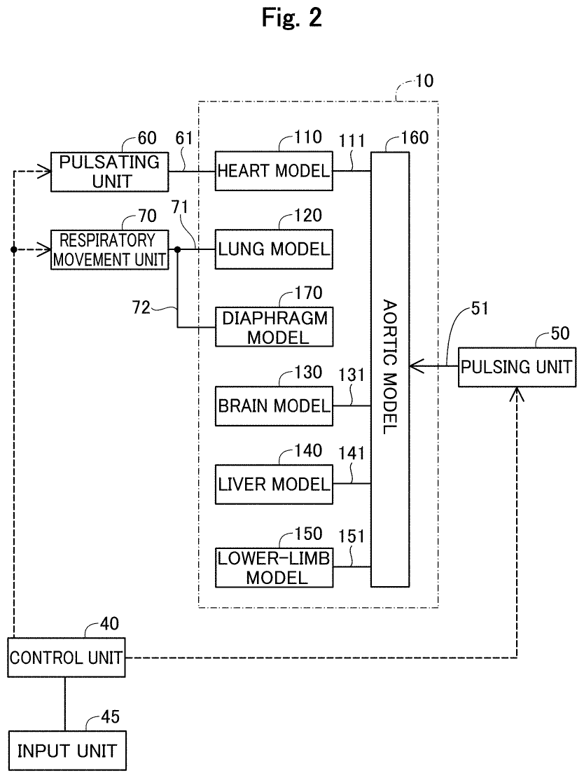

FIGS. 1 and 2 show schematic configurations of a human body simulating apparatus 1. The human body simulating apparatus 1 according to the present embodiment may be used to simulate procedures for treating or examining living-body inner cavities such as the circulatory system, the digestive system, and the respiratory system of the human body using medical devices for low invasive treatment or examination such as catheters and guide wires. The human body simulating apparatus 1 includes a model 10, a housing 20, a control unit 40, an input unit 45, a pulsing unit 50, a pulsating unit 60, and a respiratory movement unit 70.

As shown in FIG. 2, the model 10 includes a heart model 110 simulating the human heart, a lung model 120 simulating the lung, a diaphragm model 170 simulating the diaphragm, a brain model 130 simulating the brain, a liver model 140 simulating the liver, a lower-limb model 150 simulating the lower limbs, and an aortic model 160 simulating the aorta. Hereinafter, the heart model 110, the lung model 120, the diaphragm model 170, the brain model 130, the liver model 140, and the lower-limb model 150 may also be collectively called a “biological model.” The lung model 120 and the diaphragm model 170 may also be collectively called a “respiratory-organ model.” Each of the biological models except for the lung model 120 and the diaphragm model 170 is connected to the aortic model 160. The model 10 will be described in detail below.

The housing 20 includes a tank 21 and a cover 22. The tank 21 may be a substantially rectangular parallelepiped tank having an opening at the top. As shown in FIG. 1, the model 10 is placed on the bottom of the tank 21 which has been filled with a fluid to immerse the model 10 under the fluid. According to the present embodiment, water (liquid) is used as the fluid to maintain the model 10 in a wet condition as in the actual human body. It is noted that other liquids (for example, physiological saline, aqueous solutions of any compounds, and the like) may be used as the fluid. The fluid filled in the tank 21 will be uptaken into the inside of the aortic model 160 and others of the model 10 to function as a “blood simulate” which simulates blood.

The cover 22 covers the opening of the tank 21, e.g., a plate-shaped member. The cover 22 can function as a wave-cancelling plate by placing the cover 22 so that one surface of the cover 22 is brought into contact with the fluid while the other is exposed to the air. This can prevent decreased visibility due to waving of the fluid inside the tank 21. The tank 21 and the cover 22 according to the present embodiment, which are formed with a radiolucent and highly transparent synthetic resin (for example, acrylic resin), can improve the visibility of the model 10 from the outside. It is noted that the tank 21 and the cover 22 may be formed with another synthetic resin, or may be formed with different materials.

The control unit 40 includes CPU, ROM, RAM, and a storage unit, and can control the operations of the pulsing unit 50, the pulsating unit 60, and the respiratory movement unit 70 by deploying and running a computer program stored in the ROM in the RAM. The control unit 40 refers to circuitry that may be configured via the execution of computer readable instructions, and the circuitry may include one or more local processors (e.g., CPU's), and/or one or more remote processors, such as a cloud computing resource, or any combination thereof. The input unit 45 may be an interface of any kind used for a user to input information into the human body simulating apparatus 1. The input unit 45 may be, for example, a touch screen, a keyboard, an operation button, an operation dial, a microphone, or the like. Hereinafter, a touch screen is used as an illustrative example of the input unit 45.

The pulsing unit 50 is a “fluid supplying unit” which can discharge a pulsed fluid to the aortic model 160. Specifically, the pulsing unit 50 can circulate and pass a fluid in the tank 21 through the aortic model 160 of the model 10 as indicated by the open arrow in FIG. 1. The pulsing unit 50 according to the present embodiment includes a filter 55, a circulation pump 56, and a pulsing pump 57. The filter 55 is connected to an opening 210 of the tank 21 through a tubular body 31. The filter 55 can filter a fluid passing through the filter 55 to remove impurities (for example, a contrast agent used during procedures) in the fluid. The circulation pump 56 may be, for example, a non-positive displacement centrifugal pump, and can circulate a fluid coming from the tank 21 through the tubular body 31 at a constant flow rate.

The pulsing pump 57 may be, for example, a positive-displacement reciprocating pump, and can add a pulse to the fluid discharged from the circulation pump 56. The pulsing pump 57 is connected to the aortic model 160 of the model 10 through a tubular body 51 (FIG. 2). Therefore, a fluid discharged from the pulsing pump 57 is passed to the inner cavity of the aortic model 160. It is noted that a rotary pump operated at a low speed may be used as the pulsing pump 57 instead of a reciprocating pump. Further, the filter 55 and the circulation pump 56 may be omitted. The tubular body 31 and the tubular body 51 are flexible tubes formed with a synthetic resin (for example, silicone and the like) of a radiolucent soft material.

The pulsating unit 60 can cause the heart model 110 to pulsate. Specifically, the pulsating unit 60 can expand the heart model 110 by discharging a fluid into the inner cavity of the heart model 110, and can contract the heart model 110 by withdrawing the fluid from the inner cavity of the heart model 110 as indicated by the hatched arrow in FIG. 1. The pulsating unit 60 can repeat these discharging and withdrawing operations to create pulsating movements (expansion and contraction movements) of the heart model 110. A fluid which can be used with the pulsating unit 60 (hereinafter referred to as an “expansion medium”) may be a liquid like a blood simulate, or may be, for example, a gas such as air. An expansion medium is preferably an organic solvent such as benzene and ethanol or a radiolucent liquid such as water. The pulsating unit 60 may be implemented by using, for example, a positive-displacement reciprocating pump. The pulsating unit 60 is connected to the aortic model 110 of the model 10 through a tubular body 61 (FIG. 2). The tubular body 61 is a flexible tube formed with a synthetic resin (for example, silicone and the like) of a radiolucent soft material.

The respiratory movement unit 70 enables the lung model 120 and the diaphragm model 170 to simulate respiratory movements. Specifically, the respiratory movement unit 70 can discharge a fluid to an inner cavity of the lung model 120 and the diaphragm model 170 as indicated by the dot-hatched arrow in FIG. 1 to expand the lung model 120 and contract the diaphragm model 170. Further, the respiratory movement unit 70 can withdraw a fluid from the inner cavity of the lung model 120 and the diaphragm model 170 to contract the lung model 120 and relax the diaphragm model 170. The respiratory movement unit 70 can repeat these discharging and withdrawing operations to generate respiratory movements of the lung model 120 and the diaphragm model 170. A fluid which can be used with the respiratory movement unit 70 may be a liquid like a blood simulate, or may be, for example, a gas such as air. The respiratory movement unit 70 can be implemented by using, for example, a positive-displacement reciprocating pump. The respiratory movement unit 70 is connected to the lung model 120 of the model 10 through a tubular body 71, and connected to the diaphragm model 170 through a tubular body 72 (FIG. 2). The tubular bodies 71, 72 are flexible tubes formed with a synthetic resin (for example, silicone and the like) of a radiolucent soft material.

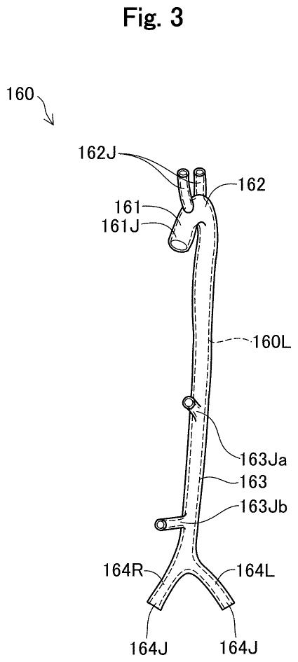

FIG. 3 shows a schematic configuration of the aortic model 160. The aortic model 160 is composed of components which simulate those of the human aorta, i.e., an ascending aorta portion 161 which simulates the ascending aorta, an aortic arch portion 162 which simulates the aortic arch, an abdominal aorta portion 163 which simulates the abdominal aorta, and a common iliac aorta portion 164 which simulates the common iliac aorta.

The aortic model 160 includes a second connection portion 161J for connection with the heart model 110 at an end portion of the ascending aorta portion 161. Similarly, the aortic model 160 includes a first connection portion 162J for connection with the brain model 130 in the vicinity of the aortic arch portion 162, a third connection portion 163Ja for connection with the liver model 140 in the vicinity of the abdominal aorta portion 163, and a pair of fourth connection portions 164J for connection with the right and left lower-limb models 150, respectively, at an end portion of the common iliac aorta portion 164. It is noted that the second connection portion 161J may be arranged at the ascending aorta portion 161 or in the vicinity thereof, and the fourth connection portions 164J may be arranged at the common iliac aorta portion 164 or in the vicinity thereof. Hereinafter, the first to fourth connection portions 161J to 164J may also be collectively called a “biological-model connection portion.” Further, the aortic model 160 includes a fluid supplying unit connection portion 163Jb for connection with the pulsing unit 50 in the vicinity of the abdominal aorta portion 163. It is noted that the fluid supplying unit connection portion 163Jb may be arranged at any location including in the vicinity of the ascending aorta portion 161, in the vicinity of the cerebral vascular model 131 (for example, the common carotid artery), and the like, but not limited to the abdominal aorta portion 163. Further, the aortic model 160 may include a plurality of fluid supplying unit connection portions 163Jb arranged at different locations.

Further, an inner cavity 160L is formed inside the aortic model 160. The inner cavity 160L has an opening for each of the aforementioned biological-model connection portions and fluid supplying unit connection portion (the first connection portion 162J, the second connection portion 161J, the third connection portion 163Ja, the pair of fourth connection portions 164J, and the fluid supplying unit connection portion 163Jb). The inner cavity 160L can function as a flow path for transporting a blood simulate (a fluid) passed from the pulsing unit 50 to the heart model 110, the brain model 130, the liver model 140, and the lower-limb models 150.

The aortic model 160 according to the present embodiment may be formed with a synthetic resin (for example, polyvinyl alcohol (PVA), silicone, and the like) of a radiolucent soft material. In particular, use of PVA is preferred in that the hydrophilicity of PVA enables a user to feel the aortic model 160 immersed under a liquid as if it were the actual human aorta in the body.

The aortic model 160 may be produced, for example, as follows. First, a mold is prepared which simulates the shape of the human aorta. The mold may be produced by, for example, 3D-printing using data of the aorta in the human model data generated by analyzing images from CT (Computed Tomography) or MRI (Magnetic Resonance Imaging) of an actual human body. The mold may be of gypsum, metal, or resin. Next, a liquefied synthetic resin material may be applied on the inner surface of the mold prepared. After cooled and cured, the synthetic resin material is demolded. In this way, the aortic model 160 having the inner cavity 160L can easily be produced.

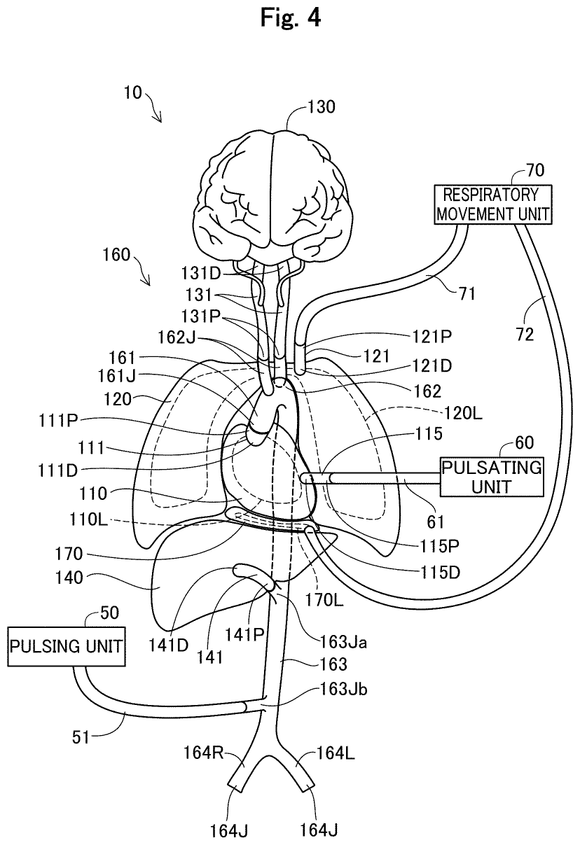

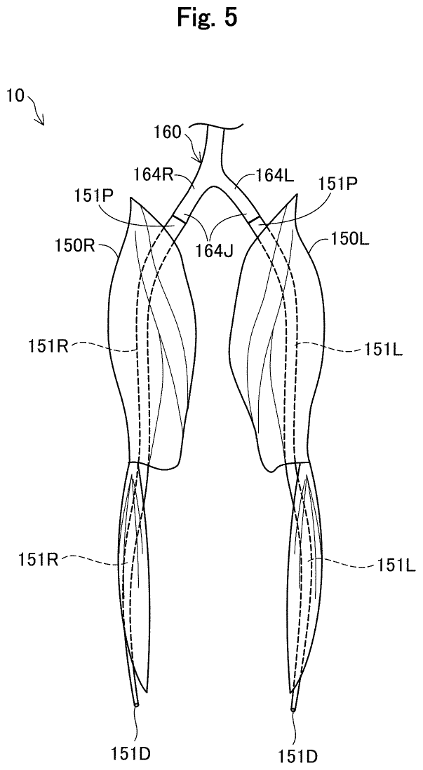

FIGS. 4 and 5 shows schematic configurations of the model 10. As shown in FIG. 4, the heart model 110 has a shape simulating the heart, and has an inner cavity 110L formed thereinside. The heart model 110 according to the present embodiment may be formed with a synthetic resin (for example, silicone and the like) of a radiolucent soft material, and may be produced by applying a synthetic resin material on the inner surface of a mold prepared based on human body model data, and then demolding it as in the aortic model 160. Further, the heart model 110 includes a tubular body 115, and is connected to a cardiovascular model 111. The cardiovascular model 111 is a tubular vascular model which simulates a part of the ascending aorta and the coronary artery, and may be formed with a synthetic resin (for example, PVA, silicone, and the like) of a radiolucent soft material. The tubular body 115 is a flexible tube formed with a synthetic resin (for example, silicone and the like) of a radiolucent soft material. The tubular body 115 is connected so that a distal end 115D is in communication with the inner cavity 110L of the heart model 110, and a proximal end 115P is in communication with the tubular body 61 leading to the pulsating unit 60.

The lung models 120 each have a shape which simulates either the right or left lung, and have one inner cavity 120L formed thereinside which is in communication with the right lung and the left lung. The lung models 120 are arranged so as to cover the right and left sides of the heart model 110. The lung models 120 may be produced using a similar material and method as in the heart model 110. The lung models 120 may be made of the same material as the heart model 110 or may be made of different materials. Further, the lung model 120 includes a tracheal model 121 which is tubular, and simulates a part of the trachea. The tracheal model 121 may be produced using a similar material as the tubular body 115 of the heart model 110. The tracheal model 121 may be made of the same material as the tubular body 115 or may be made of a different material. The tracheal model 121 is connected so that a distal end 121D is in communication with the inner cavity 120L of the lung model 120, and a proximal end 121P is in communication with the tubular body 71 leading to the respiratory movement unit 70.

The diaphragm model 170 has a shape which simulates the diaphragm, and has an inner cavity 170L formed thereinside. The diaphragm model 170 is arranged below the heart model 110 (in other words, arranged at the opposite side of the brain model 130 across the heart model 110). The diaphragm model 170 may be produced using a similar material and method as in the heart model 110. The diaphragm model 170 may be made of the same material as the heart model 110, or may be made of a different material. Further, the tubular body 72 leading to the respiratory movement unit 70 is connected to the diaphragm model 170 so that the inner cavity 170L of the diaphragm model 170 is in communication with an inner cavity of the tubular body 72.

The brain model 130 has a shape which simulates the brain, and is configured to be solid without having an inner cavity. The brain model 130 is arranged above the heart model 110 (in other words, arranged at the opposite side of the diaphragm model 170 across the heart model 110). The brain model 130 may be produced using a similar material and method as in the heart model 110. The brain model 130 may be made of the same material as the heart model 110 or may be made of a different material. Further, the brain model 130 is connected to the cerebral vascular model 131 which is a tubular vascular model simulating at least a part of the main arteries including a pair of the right and left vertebral arteries from a pair of the right and left common carotid arteries. The cerebral vascular model 131 may be produced using a similar material as the cardiovascular model 111 of the heart model 110. The cerebral vascular model 131 may be made of the same material as the cardiovascular model 111, or may be made of a different material. Further, although not shown, the cerebral vascular model 131 may simulate not only an artery but also a major vein including the superior cerebral vein and the straight sinus.

It is noted that the brain model 130 may be a complex further including a bone model which simulates the human cranium and cervical vertebrae. For example, a cranium model may have: a hard resin case simulating the parietal bone, temporal bone, occipital bone, and sphenoid bone; and a lid simulating the frontal bone. A cervical vertebrae model may have a plurality of rectangular resin bodies each having a through-hole thereinside through which a vascular model can pass. When included, the bone model may be produced with a resin having a hardness different from an organ model such as a vascular model and a brain model. For example, the cranium model may be produced with acrylic resin while a cervical vertebrae model may be produced with PVA.

The cerebral vascular model 131 is configured such that a distal end 131D is connected to the brain model 130, and a proximal end 131P is connected to the first connection portion 162J of the aortic model 160 (which corresponds to, for example, the brachiocephalic artery, the subclavian artery, or the vicinity thereof in the human body). The distal end 131D of the cerebral vascular model 131 may simulate a vertebral artery passing through the vertebrae and a different blood vessel arranged on the surface and/or in the inside of the brain model 130 (for example, the posterior cerebral artery, the middle cerebral artery), or may further simulate the posterior communicating artery, and be connected to a peripheral portion of the common carotid artery. Further, the proximal end 131P of the cerebral vascular model 131 is connected to the first connection portion 162J so that an inner cavity of the cerebral vascular model 131 is in communication with the inner cavity 160L of the aortic model 160.

The liver model 140 has a shape which simulates the liver, and is configured to be solid without having an inner cavity. The liver model 140 is arranged below the diaphragm model 170. The liver model 140 may be produced using a similar material and method as in the heart model 110. The liver model 140 may be made of the same material as the heart model 110, or may be made of a different material. Further, the liver model 140 is connected to a hepatic vascular model 141 which is a tubular vascular model simulating a part of the hepatic blood vessel. The hepatic vascular model 141 may be produced using a similar material as the cardiovascular model 111 of the heart model 110. The hepatic vascular model 141 may be made of the same material as the cardiovascular model 111, or may be made of a different material.

The hepatic vascular model 141 is configured so that a distal end 141D is connected to the liver model 140, and a proximal end 141P is connected to the third connection portion 163Ja of the aortic model 160. The distal end 141D of the hepatic vascular model 141 may simulate a different blood vessel arranged on the surface and/or the inside of the liver model 140 (for example, the hepatic artery). Further, the proximal end 141P of the hepatic vascular model 141 is connected to the third connection portion 163Ja so that an inner cavity of the hepatic vascular model 141 is in communication with the inner cavity 160L of the aortic model 160.

As shown in FIG. 5, the lower-limb model 150 includes a lower-limb model 150R for the right leg and a lower-limb model 150L for the left leg. The lower-limb models 150R and 150L, which have the same configuration except for constructive symmetry, hereinafter shall be described interchangeably as “the lower-limb model 150.” The lower-limb model 150 has a shape which simulates at least a part of the major tissues such as quadriceps and crural tibialis anterior muscle in the thigh, peroneus longus, and extensor digitorum longus muscle, and is configured to be solid without having an inner cavity. The lower-limb model 150 may be produced using a similar material and method as in the heart model 110. The lower-limb model 150 may be made of the same material as the heart model 110, or may be made of a different material. Further, the lower-limb model 150 is connected to a lower-limb vascular model 151 (the lower limb vascular models 151R, 151L) which is a tubular vascular model simulating at least a part of the main artery from a femoral artery to the dorsalis pedis artery. The lower-limb vascular model 151 may be produced using a similar material as the cardiovascular model 111 of the heart model 110. The lower-limb vascular model 151 may be made of the same material as the cardiovascular model 111, or may be made of a different material. Further, the lower-limb vascular model 151 may simulate not only arteries but also the main vein from the common iliac vein to the great saphenous vein although not shown.

The lower-limb vascular model 151 is arranged so as to extend through the inside of the lower-limb model 150 in the extending direction from the thigh toward the side of the crus. The lower-limb vascular model 151 is configured such that a distal end 151D is exposed at a lower end (which corresponds to a portion from the tarsal portion to the acrotarsium portion) of the lower-limb model 150, and a proximal end 151P is connected to the fourth connection portion 164J of the aortic model 160. Here, the proximal end 151P is connected to the fourth connection portion 164J so that an inner cavity of the lower-limb vascular model 151 is in communication with the inner cavity 160L of the aortic model 160.

It is noted that the cardiovascular model 111, the cerebral vascular model 131, the hepatic vascular model 141, and the lower-limb vascular model 151 as described above may also be collectively called a “partial vascular model.” Further, the partial vascular models and the aortic model 160 may also be collectively called a “vascular model.” These configurations enable a partial vascular model arranged on the surface of each of a biological model to simulate, for example, the posterior cerebral artery on the brain, the left and right coronary arteries on the heart, or the like. Further, these enable a partial vascular model arranged in the inside of each of a biological model to simulate, for example, the middle cerebral artery in the brain, the hepatic artery in the liver, the femoral artery in the lower limb, and the like.

In the human body simulating apparatus 1 according to the present embodiment, at least one or more biological models (the heart model 110, the lung model 120, the diaphragm model 170, the brain model 130, the liver model 140, the lower-limb model 150) can be attached to or detached from the aortic model 160 to configure the model 10 according to various aspects. A combination of the biological models (the heart model 110, the lung model 120, the diaphragm model 170, the brain model 130, the liver model 140, the lower-limb model 150) to be attached to the aortic model 160 can be appropriately changed depending on an organ required for a procedure. For example, the model 10 having the heart model 110 and the lower-limb model 150 attached can be used for simulating a procedure of the TFI (Trans-Femoral Intervention) approach of PCI with the human body simulating apparatus 1. In addition to these, all of the biological models except for the lower-limb model 150, for example, may be attached, or the heart model 110 and the lung model 120 may be attached, or the lung model 120 and the diaphragm model 170 may be attached, or the liver model 140 alone may be attached, or the lower-limb model 150 alone may be attached.

As described above, in the human body simulating apparatus 1 according to the present embodiment, a biological model (the heart model 110, the brain model 130, the liver model 140, the lower-limb model 150) which simulates a part of the inside of the human body may be connected to a biological-model connection portion (the first connection portions 162J, the second connection portion 161J, the third connection portion 163Ja, the fourth connection portion 164J) to simulate various procedures for a living-body inner cavity of each of an organ such as the circulatory system and the digestive system using a medical device such as a catheter and a guide wire depending on the connected biological model(s). Further, biological models can be detachably attached to the biological-model connection portions 161J to 164J, and thus a biological model which is not used for that procedure may also be removed and stored separately. This can improve convenience.

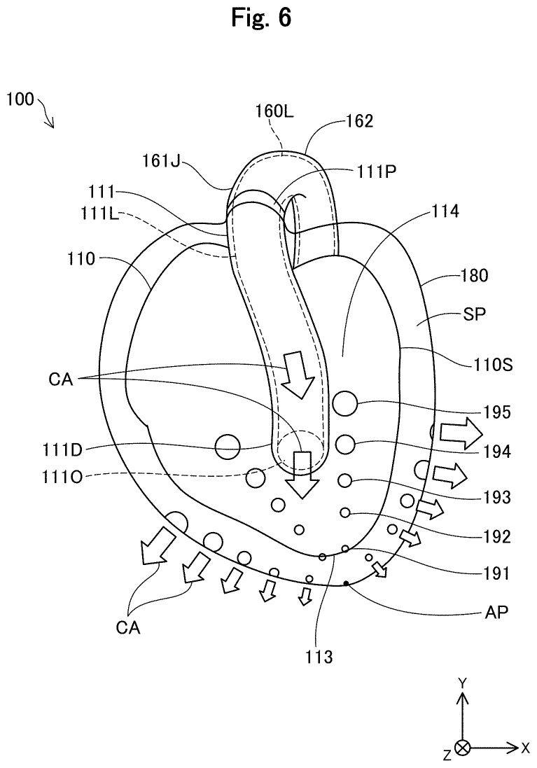

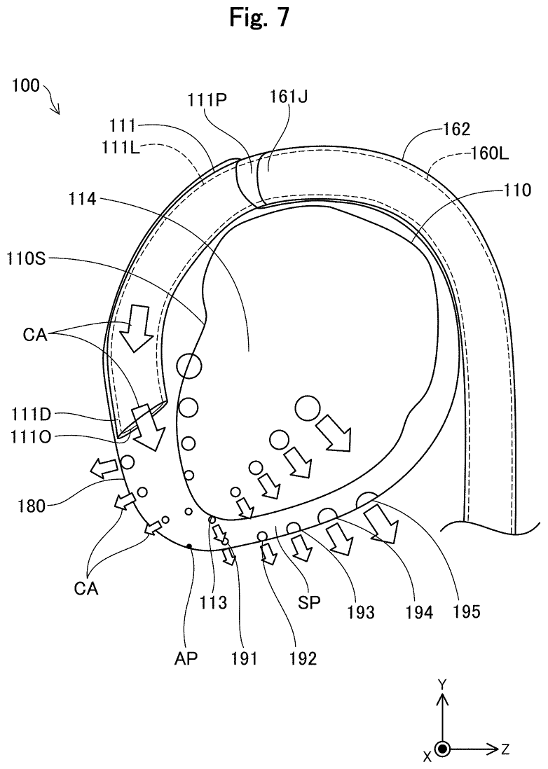

FIGS. 6 and 7 show a schematic configuration of a heart simulator 100. The heart simulator 100 further includes a pericardium member 180 in addition to the heart model 110 and the cardiovascular model 111 described with reference to FIG. 4. It is noted that FIGS. 6 and 7 omit the illustration of the tubular body 115 and the inner cavity 110L (FIG. 4) of the heart model 110 and illustrate the heart model 110 and the cardiovascular model 111 covered by the pericardium member 180 with continuous lines, for the convenience of illustration. The heart simulator 100 according to the present embodiment includes the pericardium member 180 with a configuration described later, enabling the flow of a contrast agent (X-ray images) during use of the contrast agent to simulate the flow in an actual living body where a contrast agent spreads along arterioles on the surface of the heart, and then diffuses over venules to disappear.

The XYZ axes orthogonally intersecting to each other are shown in FIGS. 6 and 7. The X-axis corresponds to the lateral direction (the width direction) of the heart model 110, and the Y-axis corresponds to the height direction of the heart model 110, and the Z-axis corresponds to the depth direction of the heart model 110. In FIGS. 6 and 7, the upper side (the +Y axis direction) corresponds to a “proximal side,” and the lower side (the −Y axis direction) corresponds to a “distal side.” In each of the constituent components of the heart simulator 100, a proximal side is also referred to as a “proximal end side,” and the distal side is also referred to a “distal end side.” Further, the end portion located on the distal side is also referred to as a “distal end” and a portion located at a distal end and in the vicinity of the distal end is also referred to as “distal end portion”. Further, the end portion located on the proximal end side is also referred to as a “proximal end” and a portion located at a proximal end and in the vicinity of the proximal end is also referred to as “proximal end portion”.

The heart model 110 has an external shape simulating the human heart, wherein a cardiac base portion 114 is formed on the proximal end side and a cardiac apex portion 113 is formed on the distal end side. The cardiovascular model 111 is arranged outside and adjacent to the heart model 110. A proximal end 111P of the cardiovascular model 111 is connected to the second connection portion 161J of the aortic model 160 so that an inner cavity 111L of the cardiovascular model 111 is in communication with the inner cavity 160L of the aortic model 160. Further, an opening 1110 in communication with the inner cavity 111L is formed at a distal end 111D of the cardiovascular model 111.

The pericardium member 180 is a saclike thin film covering the heart model 110 and the cardiovascular model 111. The pericardium member 180 may be formed with a synthetic resin (for example, polyvinyl alcohol (PVA), urethane rubber, and silicone rubber) of a radiolucent soft material. The pericardium member 180 according to the present embodiment has elasticity lower than that of the heart model 110. As shown in FIG. 7, the heart model 110 is entirely and a part of the cardiovascular model 111, which is on the distal end side, is housed in a space SP (hereafter, is also referred to as “internal space SP”) between the inner surface of the pericardium member 180 and a surface 110S of the heart model 110.

The pericardium member 180 has a plurality of through-holes 191 to 195 that are formed to penetrate the inside and outside of the pericardium member 180. Each of the through-holes 191 to 195 may be formed so that the internal space SP of the pericardium member 180 is in communication with the interior of the external tank 21. Accordingly, the internal space SP of the pericardium member 180 is, therefore, filled with a fluid in the tank 21 flowing therein through each of the through-holes 191 to 195.

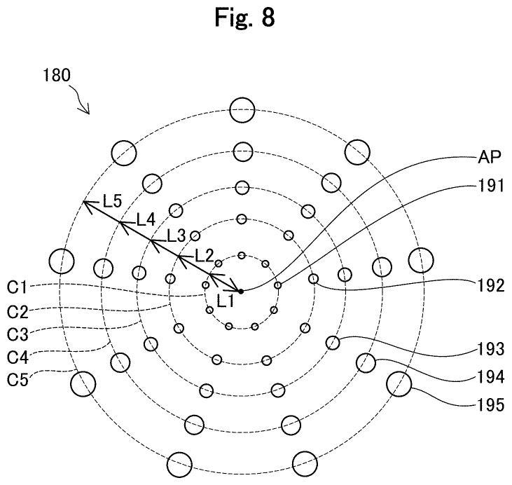

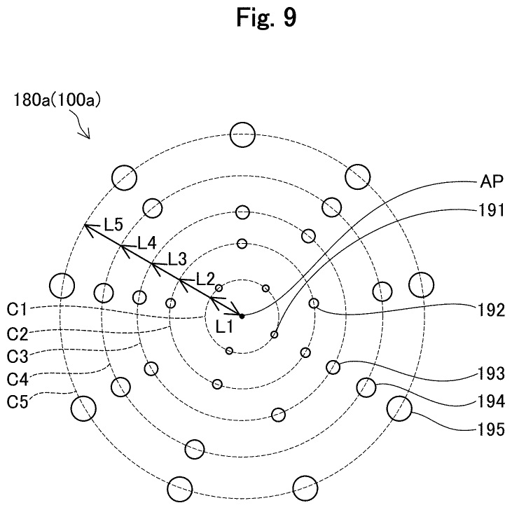

FIG. 8 illustrates a configuration of the pericardium member 180. FIG. 8 illustrates five concentric circles C1 to C5 centered at point AP (FIG. 8: circles C1 to C5 denoted with broken lines). A position in the vicinity of the point AP and the innermost circle C1 corresponds to that of the pericardium member 180 where a cardiac apex portion 113 is covered. Further, a position in the vicinity of the outermost circle C5 corresponds to that of the pericardium member 180 where a cardiac base portion 114 is covered. In other words, in FIG. 8, the pericardium member 180 moves from the position where the pericardium member 180 covers the cardiac apex portion 113 to the position where the same covers the cardiac base portion 114 as it moves away from the point AP, specifically from the circle C1 to the circle C5. The circles C1 to C5 are centered at the point AP and located at equal intervals away from the point AP, e.g., a distance between adjacent circles is the same. For example, a radius L5 of the circle C5 is 5 times greater than a radius L1 of the circle C1. Similarly, a radius L4 of the circle C4 is 4 times greater than the radius L1 of the circle C1, a radius L3 of the circle C3 is 3 times greater than the radius L1 of the circle C1, and a radius L2 of the circle C2 is twice the radius L1 of the circle C1. The same applies to the following FIGS. 9 to 13.

In the pericardium member 180, nine through-holes 191 are formed on the innermost circle C1. Each through-hole 191 is a circular pore and the opening area is smaller than that of any other through-holes 192 to 195. In the pericardium member 180, nine through-holes 192 are formed on the circle C2 located outside the circle C1. Each through-hole 192 is a circular pore and the opening area is larger than that of through-holes 191 and smaller than that of through-holes 193 to 195. In the pericardium member 180, 9 through-holes 193 are formed on the circle C3 located outside the circle C2. Each through-hole 193 is a circular pore and the opening area is larger than that of the through-holes 191 and 192 and smaller than that of the through-holes 194 and 195. In the pericardium member 180, nine through-holes 194 are formed on the circle C4 located outside the circle C3. Each through-hole 194 is a circular pore and the opening area is larger than that of the through-holes 191 to 193 and smaller than that of the through-hole 195. In the pericardium member 180, nine through-holes 195 are formed on the outermost circle C5. Each through-hole 195 is a circular pore and the opening area is larger than that of any other through-holes 191 to 194.

As described above, in the pericardium member 180 according to the present embodiment, the opening area of the plurality of through-holes 191 to 195 gradually increases from the position where the pericardium member 180 covers the cardiac apex portion 113 (in the vicinity of the point AP and the innermost circle C1) toward the position where the same covers the cardiac base portion 114 (in the vicinity of the outermost circle C5). Further, in the pericardium member 180, the plurality of through-holes 191 to 195 are arranged on the concentric circles C1 to C5 centered at the position where the point AP is covered. Since the circles C1 to C5 are evenly spaced around the point AP, the plurality of through-holes 191 to 195 arranged on the adjacent circles are also evenly spaced. Further, in the pericardium member 180 according to the present embodiment, the numbers of the plurality of through-holes 191, 192, 193, 194 and 195 arranged concentrically are the same, here nine.

Note that the radiuses L5 to L1 of the circles C5 to C1 can be arbitrarily determined. Specifically, the circles C1 to C5 and the plurality of through-holes 191 to 195 arranged on the adjacent circles do not need to be arranged at equal intervals. Further, the number of through-holes arranged on the circles C1 to C5 does not have to be the same. For example, the number of through-holes 191 arranged on the circle C1 and the number of through-holes 192 arranged on the circle C2 may be different. Similarly, the number of through-holes 193 to 195 arranged on the other circles C3 to C5 may be different from each other.

As described above, the heart simulator 100 according to the first embodiment includes the pericardium member 180 that covers the heart model 110 and the cardiovascular model 111 and has the plurality of through-holes 191 to 195 that are formed to penetrate the inside and the outside. Therefore, a contrast agent CA (white arrow) discharged from the cardiovascular model 111 is gently diluted, in a ripple pattern, with a fluid filling the internal space SP of the pericardium member 180 (the space inside the pericardium member 180 and the space outside the heart model 110 and the cardiovascular model 111), and then spreads and is discharged from the internal space SP of the pericardium member 180 to the outside of the pericardium member 180 through the plurality of through-holes 191 to 195. As a result, the heart simulator 100 according to the first embodiment enables the flow of a contrast agent CA (X-ray images) during use of the contrast agent to simulate the actual flow in the living body where a contrast agent spreads along arterioles on the surface of the heart, and then diffuses over venules to disappear.

In the actual human body, arterioles on the surface of the heart, venules, and capillary vessels gradually thicken from the cardiac apex portion to the cardiac base portion, so that a relatively large amount of a contrast agent spreads and disappears on the side of the cardiac base portion. According to the heart simulator 100 of the first embodiment, the opening area of each of the plurality of through-holes 191 to 195 of the pericardium member 180 gradually increases from the position where the pericardium member 180 covers the cardiac apex portion 113 (in the vicinity of the point AP and the innermost circle C1) of the heart model 110 toward the position where the same covers the cardiac base portion 114 (in the vicinity of the outermost circle C5). Therefore, the amount of the contrast agent CA that spreads and is then discharged from the pericardium member 180 to the outside can be gradually increased from the cardiac apex portion 113 to the cardiac base portion 114, as in the actual human body (FIG. 6 and FIG. 7: white arrows illustrated outside the pericardium member 180).

In the actual human body, arterioles on the surface of the heart, venules, and capillary vessels are laid out in a mesh pattern over the surface of the heart. According to the heart simulator 100 of the first embodiment, the plurality of through-holes 191 to 195 of the pericardium member 180 are arranged on the concentric circles C1 to C5 centered at the position (point AP) where the pericardium member 180 covers the cardiac apex portion 113 of the heart model 110 (FIG. 8). This enables the flow of the contrast agent CA that spreads and is then discharged from the pericardium member 180 to the outside to simulate the flow in an actual human body.

According to the heart simulator 100 of the first embodiment, since the pericardium member 180 is formed with a thin film having elasticity lower than that of the heart model 110, the plurality of through-holes 191 to 195 can be easily formed in the pericardium member 180. Because of the elasticity of the pericardium member 180, the cardiovascular model 111 can be maintained in a state of being pressed against the heart model 110. The cardiovascular model 111 is maintained in a state of being pressed against the heart model 110, so that the deformation of the heart model 110 (for example, pulsation by the pulsating unit 60) can be transmitted to the cardiovascular model 111, and the user's immersive feeling can be improved. Further, the cardiovascular model 111 is maintained in a pressed state against the heart model 110, and, in other words, the heart model 110, the cardiovascular model 111, and the pericardium member 180 are not fixed, so that these can be easily replaced.

### Second Embodiment

FIG. 9 illustrates a configuration of a pericardium member 180a according to a second embodiment. The heart simulator 100a according to the second embodiment includes the pericardium member 180a instead of the pericardium member 180. The pericardium member 180a is different from that of the first embodiment in the configuration of the plurality of through-holes 191 to 195.

In the pericardium member 180a, four through-holes 191 are formed on the innermost circle C1. Similarly, five through-holes 192 are formed on a circle C2 outside the circle C1, six through-holes 193 are formed on a circle C3 outside the circle C2, seven through-holes 194 are formed on a circle C4 outside the circle C3, and nine through-holes 195 are formed on the outermost circle C5. The size of each of the through-holes 191 to 195 is as described in the first embodiment. As described above, in the pericardium member 180a, the number of the plurality of through-holes 191, 192, 193, 194 and 195 arranged concentrically is different from each other, and the number of the plurality of through-holes 191 to 195 gradually increases from the position where the pericardium member 180a covers the cardiac apex portion 113 (in the vicinity of the point AP and the innermost circle C1) toward the position where the same covers the cardiac base portion 114 (in the vicinity of the outermost circle C5).

As described above, the configuration of the plurality of through-holes 191 to 195 formed in the pericardium member 180a can be variously changed. For example, all (FIG. 9) or at least some of the numbers of the plurality of through-holes 191, 192, 193, 194, and 195 arranged concentrically may differ from each other. The heart simulator 100a according to the second embodiment can also exert similar effects as in the first embodiment above. According to the heart simulator 100a of the second embodiment, the number of the plurality of through-holes 191 to 195 arranged concentrically gradually increases from the position where the pericardium member 180a covers the cardiac apex portion 113 of the heart model 110 (in the vicinity of the point AP and the innermost circle C1) toward the position where the same covers the cardiac base portion 114 (in the vicinity of the outermost circle C5). Therefore, the amount of the contrast agent CA that spreads and is then discharged from the pericardium member 180 to the outside can be gradually increased from the cardiac apex portion 113 to the cardiac base portion 114, as in the actual human body.

### Third Embodiment

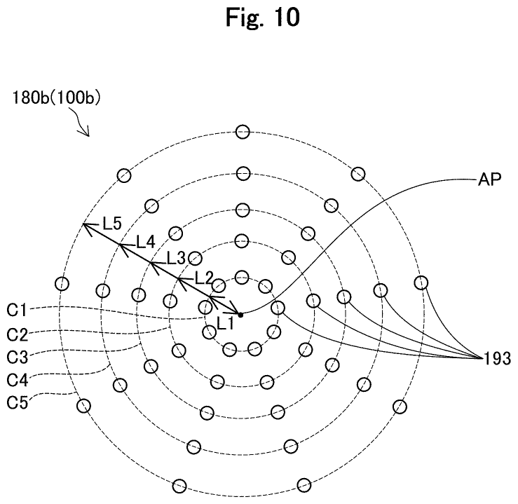

FIG. 10 illustrates a configuration of a pericardium member 180b according to a third embodiment. The heart simulator 100b according to the third embodiment includes the pericardium member 180b instead of the pericardium member 180. The pericardium member 180b includes a plurality of through-holes 193, but does not include the through-holes 191, 192, 194 and 195 described in the first embodiment.

In the pericardium member 180b, nine through-holes 193 are formed on the innermost circle C1. Similarly, nine through-holes 193 are formed on the circle C2 outside the circle C1, on the circle C3 outside the circle C2, on the circle C4 outside the circle C3, and on the outermost circle C5, respectively. The size of each of the through-holes 193 is as described in the first embodiment. In other words, the pericardium member 180b has the through-holes 193 with the same size and shape arranged on the concentric circles, and the numbers of the plurality of the through-holes arranged concentrically are the same.

As described above, the configuration of the plurality of through-holes 193 formed in the pericardium member 180b can be variously changed, and, in the pericardium member 180b, the through-holes 193 having the same size and shape may be arranged concentrically, and the numbers of the plurality of through-holes 193 arranged concentrically may be the same. In FIG. 10, the through-holes 193 having an opening area larger than that of the through-holes 191 and 192 and smaller than that of the through-holes 194 and 195 are illustrated, but in the pericardium member 180b, through-holes having an arbitrary opening area may be formed. The heart simulator 100b according to the third embodiment can also exert similar effects as in the first embodiment above.

### Fourth Embodiment

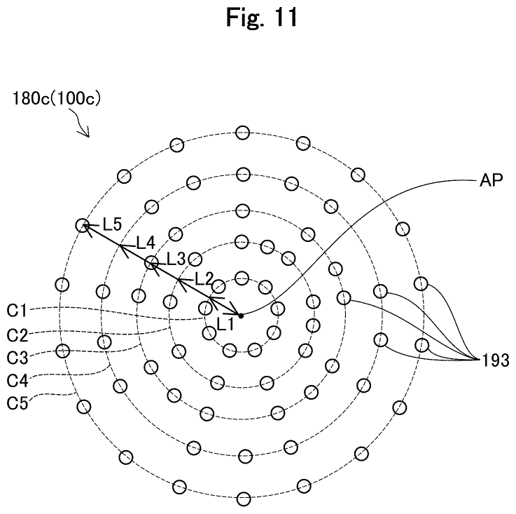

FIG. 11 illustrates a configuration of a pericardium member 180c according to a fourth embodiment. The heart simulator 100c according to the fourth embodiment includes the pericardium member 180c instead of the pericardium member 180. The pericardium member 180c includes a plurality of through-holes 193, but does not include the through-holes 191, 192, 194 and 195 described in the first embodiment, e.g., all through-holes have a same opening area, here the mid-range open area of through-holes 193.

In the pericardium member 180c, nine through-holes 193 are formed on the innermost circle C1. Similarly, eleven through-holes 193 are formed on a circle C2 outside the circle C1, twelve through-holes 193 are formed on a circle C3 outside the circle C2, fourteen through-holes 193 are formed on a circle C4 outside the circle C3, and eighteen through-holes 193 are formed on the outermost circle C5. In other words, in the pericardium member 180c, through-holes 193 having the same size and the same shape are arranged on concentrically, and, the number of the plurality of through-holes 193 arranged concentrically gradually increases from the position (in the vicinity of the point AP and the innermost circle C1) where the pericardium member 180c covers the cardiac apex portion 113 toward the position (in the vicinity of the outermost circle C5) where the same covers the cardiac base portion 114.

As described above, the configuration of the plurality of through-holes 193 formed in the pericardium member 180c can be variously changed, and, in the pericardium member 180c, the through-holes 193 having the same size and the same shape may be arranged concentrically, and the numbers of the plurality of through-holes 193 arranged concentrically may be different. In FIG. 11, the through-holes 193 having an opening area larger than that of the through-holes 191 and 192 and smaller than that of the through-holes 194 and 195 are illustrated, but in the pericardium member 180c, through-holes having an arbitrary opening area may be formed. Such heart simulator 100c according to the fourth embodiment can also exert similar effects as in the first embodiment.

### Fifth Embodiment

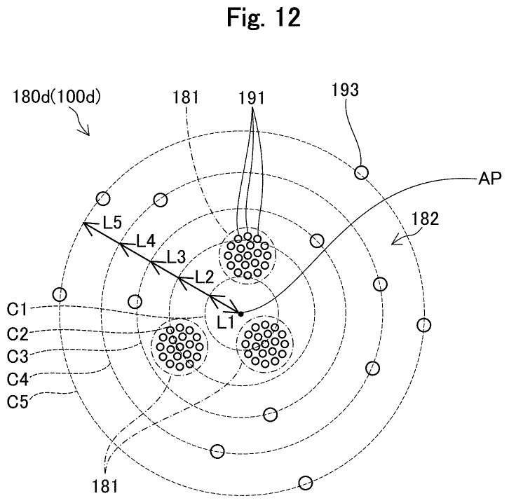

FIG. 12 illustrates a configuration of a pericardium member 180d according to a fifth embodiment. A heart simulator 100d according to the fifth embodiment includes the pericardium member 180d instead of the pericardium member 180. The pericardium member 180d has a plurality of regions (a first region 181 and a second region 182) that are different in terms of the density of the plurality of through-holes 191 and 193.

The first region 181 is a region in the pericardium member 180d in which the density of through-holes formed in the pericardium member 180d is relatively high compared to other regions in the pericardium member 180d. In the illustrated example, the region (FIG. 12: alternate long and short dash line frame) in which a plurality of through-holes 191 are densely formed corresponds to the first region 181. The first region 181 is provided at a position on the cardiac apex portion 113 side of the heart model 110 (in the vicinity of the inner circles C1 and C2). The second region 182 means a region in the pericardium member 180d in which the density of through-holes formed in the pericardium member 180d is relatively low relative to the first region 181. In the illustrated example, the region other than the first region 181 corresponds to the second region 182. The plurality of through-holes 193 are formed in the second region 182.

As described above, the configurations of the plurality of through-holes 191 and 193 formed in the pericardium member 180d can be variously changed, and the pericardium member 180d may include the first region 181 having a relatively high density of through-holes, and the second region 182 having relatively low density of through-holes. Further, in the first region 181 having relatively high density of through-holes, through-holes 191 having a smaller opening area than that of the second region 182 may be formed. Further, the opening areas of the through-holes in the first region 181 and the second region 182 may be the same, and in the first region 181, through-holes having a larger opening area than the second region 182. Such heart simulator 100d according to the fifth embodiment can also exert similar effects as in the first embodiment above.

In the actual human body, arterioles on the surface of the heart, venules, and capillary vessels, specifically, each distal end of the arterioles and venules (ends on the cardiac apex portion side) is connected by capillary vessels on the cardiac apex portion side. According to the heart simulator 100d of the fifth embodiment, the pericardium member 180d is provided with a first region 181 where the opening area of the plurality of through-holes is smaller than that of the plurality of through-holes 193 provided at the cardiac base portion 114, and the density of the through-holes 191 is relatively high, at the position (in the vicinity of the inner circles C1 and C2) on the cardiac apex portion side of the heart model 110. Therefore, the capillary vessels on the surface of the heart can be simulated by the first regions 181, enabling the flow of the contrast agent CA during use of the contrast agent to further simulate the flow in an actual living body.

### Sixth Embodiment

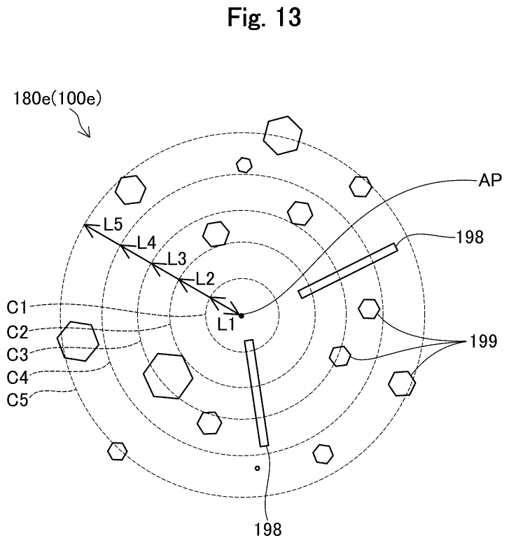

FIG. 13 illustrates a configuration of a pericardium member 180e according to a sixth embodiment. The heart simulator 100e according to the sixth embodiment includes the pericardium member 180e instead of the pericardium member 180. The pericardium member 180e has a plurality of through-holes 198 and 199 formed therein instead of the plurality of through-holes 191 to 195. The through-holes 198 are long-shaped (slit-shaped) through-holes. The through-holes 199 are polygonal (hexagonal in the illustrated example) through-holes. Each of the through-holes 198 and 199 has a different opening area. Further, each of the through-holes 198 and 199 is not arranged on the concentric circles C1 to C5, but is formed at random positions on the pericardium member 180e. Additionally, opening areas of one or more through-holes 199 may be different from each other and may be randomly situated, e.g., not necessarily increasing away from the point AP.

As described above, the configurations of the plurality of through-holes 198 and 199 formed in the pericardium member 180e can be variously changed, and the plurality of through-holes 198 and 199 may each have a different shape and different opening area. Further, the plurality of through-holes 198 and 199 are not arranged concentrically, but are formed at random positions on the pericardium member 180e. Such heart simulator 100e according to the sixth embodiment can also exert similar effects as in the first embodiment.

### Seventh Embodiment

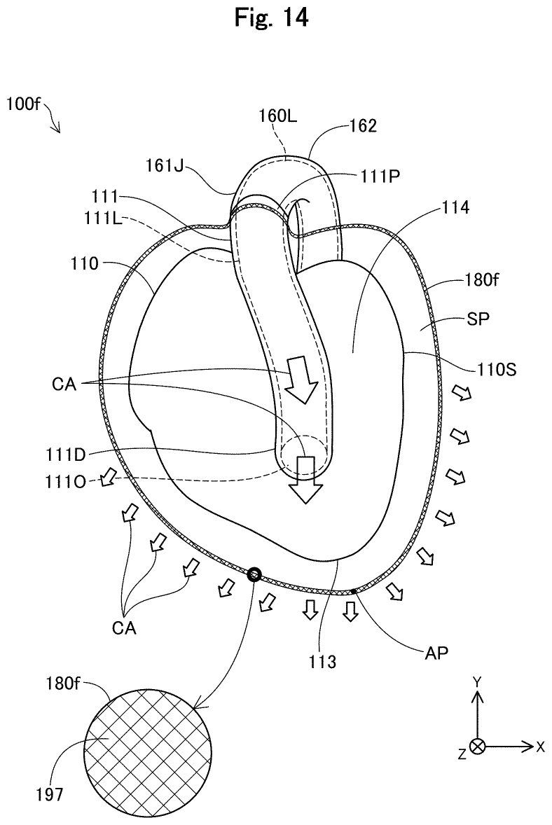

FIG. 14 shows a schematic configuration of a heart simulator 100f according to a seventh embodiment. The heart simulator 100f according to the seventh embodiment includes the pericardium member 180f instead of the pericardium member 180. The pericardium member 180f is a saclike thin film covering the heart model 110 and the cardiovascular model 111 and is formed with a porous body. The pericardium member 180f can be formed with, for example, a foam body such as a silicone foam, a urethane foam, a rubber sponge, and an acrylic foam. As shown in the enlarged view shown in the lower part of FIG. 14, pores 197 of the porous body constituting the pericardium member 180f function as a plurality of through-holes penetrating the inside and outside of the pericardium member 180f.

As described above, the configuration of the pericardium member 180f can be variously changed, and may be configured using a porous body having pores 197 instead of forming the through-holes 191 to 195 in the thin film. In such heart simulator 100f according to the seventh embodiment, a contrast agent CA (white arrow) discharged from the cardiovascular model 111 is gently diluted in a ripple pattern with a fluid filling the internal space SP of the pericardium member 180f, and then spreads and is discharged from the internal space SP of the pericardium member 180f to the outside of the pericardium member 180f through the plurality of through-holes (pores 197). As a result, the heart simulator 100f according to the seventh embodiment can also exert similar effects as in the first embodiment. Further, according to the heart simulator 100f of the seventh embodiment, the pericardium member 180f can be easily formed.

### Eighth Embodiment

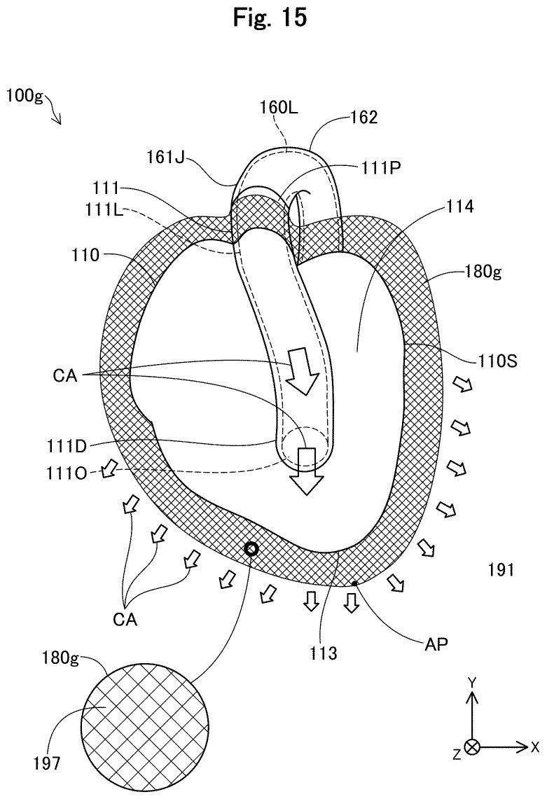

FIG. 15 shows a schematic configuration of a heart simulator 100g according to an eighth embodiment. The heart simulator 100g according to the eighth embodiment includes a pericardium member 180g instead of the pericardium member 180. The pericardium member 180g is a layer of a porous body provided to cover the surface of the heart model 110 and the cardiovascular model 111. In the example of FIG. 15, the inner surface of the pericardium member 180g is in contact with the surface 110S of the heart model 110, and the internal space SP (FIG. 6) described in the first embodiment is not formed. The pericardium member 180g can be formed of, for example, a foam such as silicon foam, urethane foam, rubber sponge, or acrylic foam, as in the seventh embodiment. As shown in the enlarged view shown in the lower part of FIG. 15, pores 197 of the porous body constituting the pericardium member 180g function as a plurality of through-holes penetrating the inside and outside of the pericardium member 180g.

As described above, the configuration of the pericardium member 180g can be variously changed, and the pericardium member 180g may be configured such that it includes no internal space SP described in the first embodiment, wherein the inner surface of the pericardium member 180g and the surface 110s of the heart model 110 come into contact with each other. Even in the heart simulator 100g according to the eighth embodiment, the contrast agent CA (white arrow) discharged from the cardiovascular model 111 diffuses through the pores 197 of the pericardium member 180g, and is then discharged to the outside of the pericardium member 180g. As a result, the heart simulator 100g according to the eighth embodiment can also exert similar effects as in the first embodiment. Further, according to the heart simulator 100g of the eighth embodiment, the pericardium member 180g can be easily formed.

### MODIFICATION EXAMPLES OF THE EMBODIMENTS

The disclosed embodiments are not limited to the above-described embodiments, and can be implemented in various aspects without departing from the gist thereof. For example, the following modification examples are also possible.

### Modification Example 1

The aforementioned first to eighth embodiments show examples of the configuration of the human body simulating apparatus 1. However, various modifications may be made to the configuration of the human body simulating apparatus. For example, the human body simulating apparatus does not need to include at least one of the tanks and the cover for covering the tank. For example, the human body simulating apparatus may include an input unit by a means other than a touch screen (for example, sound, an operation dial, a button, and the like).

### Modification Example 2

The aforementioned first to eighth embodiments show examples of the configurations of the model 10. However, various modifications may be made to the configurations of the model. For example, the aortic model does not need to include at least a part of the first to the fourth connection portions. For example, the arrangement of the first to fourth connection portions described above in the aortic model may be altered appropriately. The first connection portion does not need to be arranged at the aortic arch or in the vicinity thereof. Similarly, the second connection portion does not need to be arranged at the ascending aorta or in the vicinity thereof. The third connection portion does not need to be arranged at the abdominal aorta or in the vicinity thereof. The fourth connection portion does not need to be arranged at the common iliac aorta or in the vicinity thereof. For example, any number of biological-model connection portions may be used in the aortic model. A new biological-model connection portion for connecting a biological model not mentioned above (for example, a stomach model, a pancreas model, a kidney model, and the like) may be included.

For example, the model does not need to include at least a part of the heart model, the lung model, the brain model, the liver model, the lower-limb model, and the diaphragm model. When the lung model and the diaphragm model are omitted, the respiratory movement unit can also be omitted. For example, the model may be configured as a complex further including a bone model simulating at least a portion of a human bone such as rib, sternum, thoracic vertebra, lumbar vertebra, femur, and neckbone. For example, the configurations of the aforementioned heart model, lung model, brain model, liver model, lower-limb model, and diaphragm model may be altered appropriately. For example, the inner cavity of the heart model and the pulsating unit which discharges a fluid to the inner cavity of the heart model may be omitted (FIG. 4). The lung model may include separate inner cavities disposed at each of the right and left lungs (FIG. 4). The lower-limb model may further include a skin model which covers femur muscle (FIG. 5).

### Modification Example 3

The aforementioned first to eighth embodiments show examples of the configurations of the heart simulators 100 and 100a to 100f. However, various modifications can be made to the configurations of the heart simulators. For example, the heart simulator may be implemented alone, independent from the other configurations described in FIGS. 4 and 5 (other models, control unit, pulsing unit, pulsating unit, respiratory movement unit, input unit, and tank, etc.). For example, at least one of a heart model and a cardiovascular model included in the heart simulator may have a model simulating a healthy heart or cardiac blood vessel and a model simulating a heart or cardiac blood vessel having a lesion site, which may be interchangeably attached. For example, at least a part of the heart model, the cardiovascular model, and the pericardium member may be fixed to each other. In this case, for example, it can be fixed by using a band-shaped fixing member formed of a synthetic resin (for example, silicon or the like) made of a radiolucent soft material.

For example, the cardiovascular model may include a model simulating a vein in addition to models simulating a part of the ascending aorta and the coronary artery. For example, the cardiovascular model may have a shape that simulates a human coronary artery or a part of a coronary artery. In this case, for example, the cardiovascular model may be configured such that the inner cavity of the model may be branched into a plurality of flow paths so as to enable the diffusion of the fluid on the surface of the heart model.

### Modification Example 4

The aforementioned first to eighth embodiments show examples of the configurations of the pericardium members 180 and 180a to 180g. However, various modifications can be made to the configurations of the pericardium members. For example, the pericardium member may cover at least a part of the heart model, instead of covering the entire heart model. In this case, for example, the vicinity of the cardiac apex portion of the heart model may be covered with a pericardium member, and the vicinity of the cardiac base portion of the heart model may be exposed. For example, the pericardium member may be configured to be removable with respect to the heart model and the cardiovascular model. In this case, a plurality of pericardium members are prepared in advance depending on the ability to discharge a contrast agent according to the health conditions and age, and these may be configured to be replaceable.

### Modification Example 5

The configurations of the human body simulating apparatuses and the heart simulators according to the first to the eighth embodiments and the configurations of the human body simulating apparatuses and the heart simulators according to the modification examples 1 to 4 may be combined in an appropriate manner. For example, the through-holes having the shape described in the sixth embodiment may be adopted in the heart simulators according to the second to fifth embodiments.

In the above, the present aspects are described based on embodiments and variations. However, the embodiments of the aforementioned aspects are provided merely for clear understanding of the present aspects, and should not be construed as limiting to the present aspects. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. The present aspects can be altered or modified without departing from their spirit thereof and claims. The present aspects include any equivalents thereto. Further, the technical features thereof, if not indicated as essential in the present specification, may be appropriately deleted.

### DESCRIPTION OF REFERENCE NUMERALS

1 . . . Human body simulating apparatus
10 . . . Model
20 . . . Housing
21 . . . Tank
22 . . . Cover
31 . . . Tubular body
40 . . . Control unit
45 . . . Input unit
50 . . . Pulsing unit
51 . . . Tubular body
55 . . . Filter
56 . . . Circulation pump
57 . . . Pulsing pump
60 . . . Pulsating unit
61 . . . Tubular body
70 . . . Respiratory movement unit
71 . . . Tubular body
72 . . . Tubular body
100, 100a-g . . . Heart simulator
110 . . . Heart model
111 . . . Cardiovascular model
115 . . . Tubular body
120 . . . Lung model
121 . . . Tracheal model
130 . . . Brain model
131 . . . Cerebral vascular model
140 . . . Liver model
141 . . . Hepatic vascular model
150, 150L,R . . . Lower-limb model
151, 151L,R . . . Lower limb vascular model
160 . . . Aortic model
161 . . . Ascending aorta portion
161J . . . Second connection portion
162 . . . Aortic arch portion
162J . . . First connection portion
163 . . . Abdominal aorta portion
163Ja . . . Third connection portion
163Jb . . . Fluid supplying unit connection portion
164 . . . Common iliac aorta portion
164J . . . Fourth connection portion
170 . . . Diaphragm model
180, 180a-g . . . Pericardium member
181 . . . First region
182 . . . Second region
191-195, 198, 199 . . . Through-hole
197 . . . Pore

What is claimed is:

1. A heart simulator, comprising:
a heart model simulating the heart and having a cardiac apex portion and a cardiac base portion;
a cardiovascular model arranged outside the heart model; and
a pericardium member covering the heart model and the cardiovascular model, wherein
the pericardium member has a plurality of through-holes that penetrate the inside and the outside of the pericardium member.

2. The heart simulator according to claim 1, wherein
in the pericardium member, an opening area of each of the through-holes increases from the position where the pericardium member covers the cardiac apex portion of the heart model toward the cardiac base portion.

3. The heart simulator according to claim 2, wherein
in the pericardium member, the plurality of through-holes are arranged on concentric circles centered at the position where the pericardium member covers the cardiac apex portion of the heart model, and
the number of the plurality of through-holes arranged concentrically increases from the position where the pericardium member covers the cardiac apex portion of the heart model toward the cardiac base portion.

4. The heart simulator according to claim 3, wherein
the pericardium member has a plurality of regions having different densities of the plurality of through-holes, and,
the pericardium member has regions where an opening area of the plurality of through-holes is smaller than that of the plurality of through-holes provided at the cardiac base portion and densities of the through-holes are higher than that of the plurality of through-holes provided at the cardiac base portion, at the position on a side of the cardiac apex portion side of the heart model.

5. The heart simulator according to claim 4, wherein
the pericardium member is formed with a thin film having elasticity lower than that of the heart model.

6. The heart simulator according to claim 1, wherein
the pericardium member is formed with a porous body, and
the plurality of through-holes are pores of the porous body.

7. The heart simulator according to claim 5, wherein
the blood simulate discharged from the cardiovascular model is discharged to the outside through the plurality of through-holes.

8. The heart simulator according to claim 3, wherein the concentric circles are evenly spaced.

9. The heart simulator according to claim 1, wherein each of the plurality of through-holes has a same shape.

10. The heart simulator according to claim 9, wherein the same shape is a circular shape.

11. The heart simulator according to claim 1, wherein each of the plurality of through-holes has a same opening area.

12. The heart simulator according to claim 1, wherein at least two of the plurality of through-holes has a different shape.

13. The heart simulator according to claim 1, wherein the plurality of through-holes are randomly positioned around the cardiac apex portion.

14. The heart simulator according to claim 1, wherein the plurality of through-holes having different opening areas are randomly positioned around the cardiac apex portion.

15. The heart simulator according to claim 1, wherein
in the pericardium member, the plurality of through-holes are arranged on concentric circles centered at the position where the pericardium member covers the cardiac apex portion of the heart model, and
the number of the plurality of through-holes arranged concentrically increases from the position where the pericardium member covers the cardiac apex portion of the heart model toward the cardiac base portion.

16. The heart simulator according to claim 1, wherein
the pericardium member has a plurality of regions having different densities of the plurality of through-holes, and,
the pericardium member has regions where an opening area of the plurality of through-holes is smaller than that of the plurality of through-holes provided at the cardiac base portion and densities of the through-holes are higher than that of the plurality of through-holes provided at the cardiac base portion, at the position on the cardiac apex portion side of the heart model.

17. The heart simulator according to claim 1, wherein
the pericardium member is formed with a thin film having elasticity lower than that of the heart model.

18. The heart simulator according to claim 1, wherein
the pericardium member is formed with a porous body, and
the plurality of through-holes are pores of the porous body.

19. The heart simulator according to claim 1, wherein
the blood simulate discharged from the cardiovascular model is discharged to the outside through the plurality of through-holes.

20. A pericardium member to cover a heart model and a cardiovascular model, the pericardium member comprising:
a plurality of through-holes that penetrate the inside and the outside of the pericardium member such that an amount of fluid that spreads and is then discharged from the pericardium member to the outside is increased from a cardiac apex portion to a cardiac base portion of the heart model.
